(19) United States
(12) Patent Application Publication
(10) Pub. No.: US 2003/0100905 A1
(43) Pub. Date: May 29, 2003

(54) Ligamentum teres femoris cutter

(76) Inventor: Dana C. Mears, Glen Arm, MD (US)

Correspondence Address: JOHN F. HOFFMAN, BAKER & DANIELS, 111 EAST WAYNE STREET, SUITE 800, FORT WAYNE, IN, 46802, US

(21) Appl. No.: 09/994,492

(22) Filed: Nov. 26, 2001

(51) Int. Cl. A61B 17/14

(52) U.S. Cl. 606/81

(57) ABSTRACT

A ligamentum teres femoris cutter configured for severing the ligamentum teres femoris prior to dislocating the femoral head from the acetabulum. The ligamentum teres femoris cutter includes a curved head for insertion between the femoral head and the acetabulum. The curved head of the ligamentum teres femoris includes a distal portion having a blade spanning a pair of joint spacers having a thickness whereby the spacing between the femoral head and the acetabulum is extended to tension the ligamentum teres femoris to facilitate severing the tendon.

### BACKGROUND OF THE INVENTION

1. Field of the Invention

The present invention relates to a surgical instrument operable to sever the ligamentum teres femoris. More particularly, the present invention relates to an instrument operable to sever the ligamentum teres femoris with the femoral head seated in the acetabulum.

2. Description of the Related Art

The ligamentum teres femoris connects the head of the femur to the acetabulum. During certain orthopedic procedures, including, e.g., a total hip arthroplasty, the ligamentum teres femoris must be severed. For the purpose of the following discussion, a total hip arthroplasty is defined as a replacement of the femoral head with or without the use of a separate acetabular component. During a total hip arthroplasty, the ligamentum teres femoris must be severed to allow for removal of the femoral head. Conventionally, to remove the femoral head during a total hip arthroplasty, the femoral head is dislocated from the acetabulum, and the ligamentum teres femoris is thereafter severed with a sharp instrument.

Increasingly, orthopedic procedures for replacing all or a portion of a joint are becoming less invasive, i.e., they are performed through smaller wounds. Conventionally, procedures utilized to prepare the bone and seat the implants to effect joint replacement are open procedures. For the purpose of this discussion, the term open procedure will refer to a procedure wherein an incision is made through the skin and the underlying tissue to fully expose a large portion of the particular joint surface. After the initial incision in the skin, the internal wound may be enlarged to fully expose the areas to be prepared. While an open approach provides surgeons with an excellent view of the bone surface, the underlying damage to the soft tissue, including the muscles, can lengthen a patient's rehabilitation time after surgery. While the implants may be well fixed at the time of surgery, it may take several weeks or perhaps months before the soft tissues violated during surgery can fully heal.

Less invasive, i.e., non-open procedures for replacing all or a portion of joint have a shorter recovery time relative to open procedures, and, therefore, are favored. With respect to a total hip arthroplasty, an instrument allowing the ligamentum teres femoris to be severed while the femoral head is positioned in the acetabulum would facilitate performance of a joint replacement in a non-open procedure, since the femoral head would not need to be dislocated from the acetabulum to sever the ligamentum teres femoris. A minimally invasive total hip arthroplasty is disclosed in U.S. patent application Ser. No. 09/558,044 assigned to the assignee of the present invention, the disclosure of which is hereby explicitly incorporated by reference.

What is needed in the art is a ligamentum teres femoris cutter for severing the ligamentum teres femoris prior to dislocating the femoral head from the acetabulum.

### SUMMARY OF THE INVENTION

The present invention provides an improved ligamentum teres femoris cutter advantageously configured for severing the ligamentum teres femoris prior to dislocating the femoral head from the acetabulum. The ligamentum teres femoris cutter of the present invention includes a curved head for insertion between the femoral head and the acetabulum. The curved head of the ligamentum teres femoris cutter of the present invention includes a distal portion having a thickness whereby the spacing between the femoral head and the acetabulum is extended to tension the ligamentum teres femoris to facilitate severing the same.

### BRIEF DESCRIPTION OF THE DRAWINGS

The above-mentioned and other features and advantages of this invention, and the manner of attaining them, will become more apparent and the invention itself will be better understood by reference to the following description of an embodiment of the invention taken in conjunction with the accompanying drawings, wherein:

FIG. 1 is a plan view of a ligamentum teres femoris cutter in accordance with the present invention;

FIG. 2 is a second plan view thereof;

FIG. 3 is a sectional view thereof;

FIG. 4 is a partial plan view of the distal end of the head of a ligamentum teres femoris cutter in accordance with the present invention;

FIG. 5 is a sectional view of the distal end of the head of the ligamentum teres femoris cutter of the present invention;

FIG. 6 is a plan view of an alternative embodiment of a ligamentum teres femoris cutter in accordance with the present invention;

FIG. 7 is an anterior elevational view of a hip joint illustrating insertion of a ligamentum teres femoris cutter in accordance with the present invention;

FIG. 8 is an anterior elevational view of a hip joint illustrating a ligamentum teres femoris cutter of the present invention inserted between the femoral head and the acetabulum; and

FIG. 9 is a partial anterior elevational view illustrating the ligamentum teres femoris cutter of the present invention positioned to sever the ligamentum teres femoris.

Corresponding reference characters indicate corresponding parts throughout the several views. Although the drawings represent embodiments of the present invention, the drawings are not necessarily to scale and certain features may be exaggerated in order to better illustrate and explain the present invention. The exemplifications set out herein illustrate embodiments of the invention, in one form, and such exemplifications are not to be construed as limiting the scope of the invention in any manner.

### DETAILED DESCRIPTION OF THE INVENTION

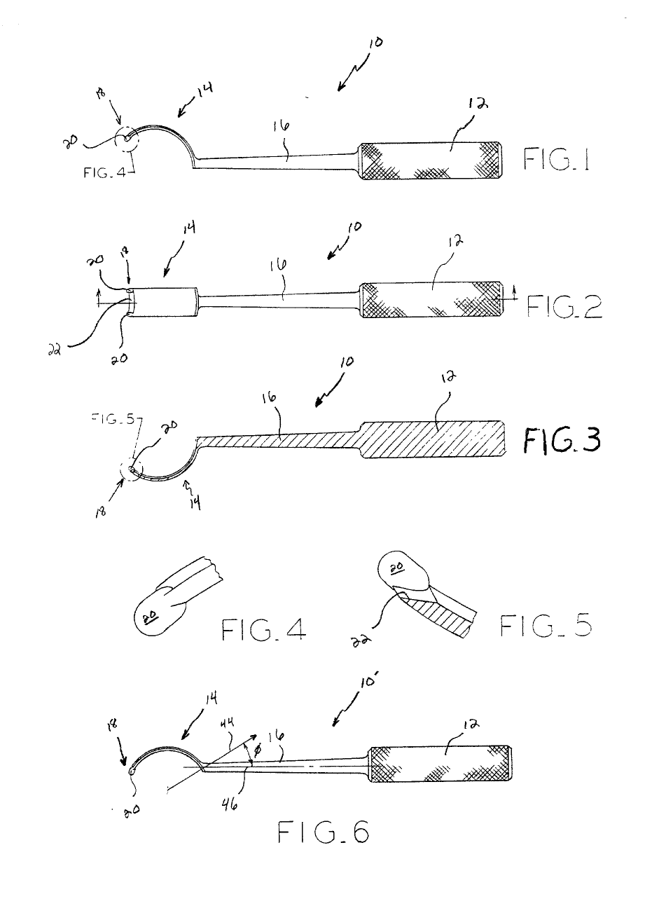

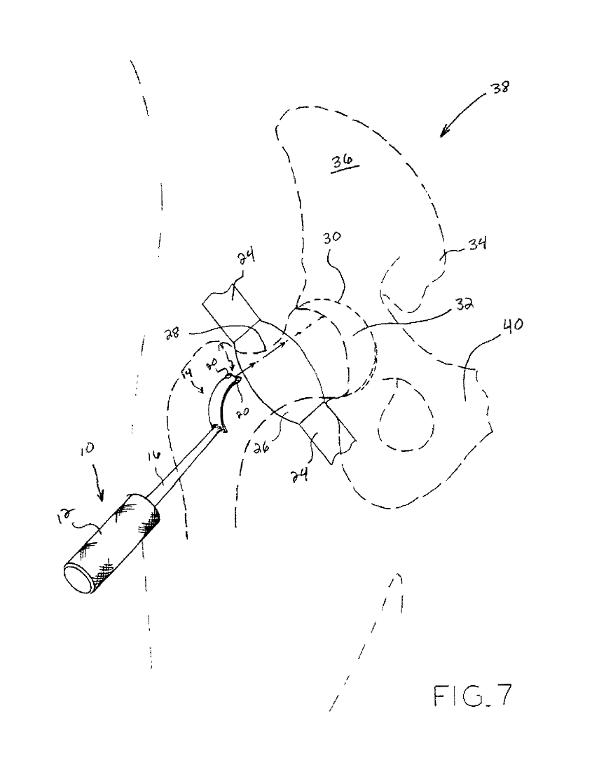

Ligamentum teres femoris cutter 10, illustrated in FIGS. 1-5, includes handle 12, tapered extension 16, and head 14 having distal end 18 with joint spacers 20 connected thereto. Blade 22 spans joint spacers 20 and forms a distal cutting surface of ligamentum teres femoris cutter 10. As illustrated in FIG. 5, blade 22 is recessed from the distal-most portion of joint spacers 20. Because blade 22 is recessed from joint spacers 20, joint spacers 20 prevent objects from contacting blade 22, thus protecting both blade 22 and, e.g., the hands of the surgical staff.

Handle 12 is formed to facilitate a firm grip of ligamentum teres femoris cutter 10 in use. In one exemplary embodiment, handle 12 is knurled to improve the grip of a surgeon's soiled glove thereon. In alternative embodiments, handle 12 may have an inconsistent cross section throughout its length, e.g., having an hourglass or an oval shaped profile to facilitate a firm grip of ligamentum teres femoris cutter 10.

Joint spacers 20 have a thickness, whereby joint spacers 20 are operable to extend the joint spacing between the femoral head and the acetabulum of a hip joint and thereby tension the ligamentum teres femoris to facilitate severing thereof. Typically, joint spacers 20 have a thickness greater than the thickness of head 13 of ligamentum teres femoris cutter 10. In one exemplary embodiment, joint spacers 20 have a thickness of about 31.25 millimeters (0.125 inches). Furthermore, joint spacers 20 may be offset from head 14 of ligamentum teres femoris cutter 10 so that joint spacers 20 cooperate with head 14 to extend the joint spacing between the femoral head and the acetabulum of a hip joint.

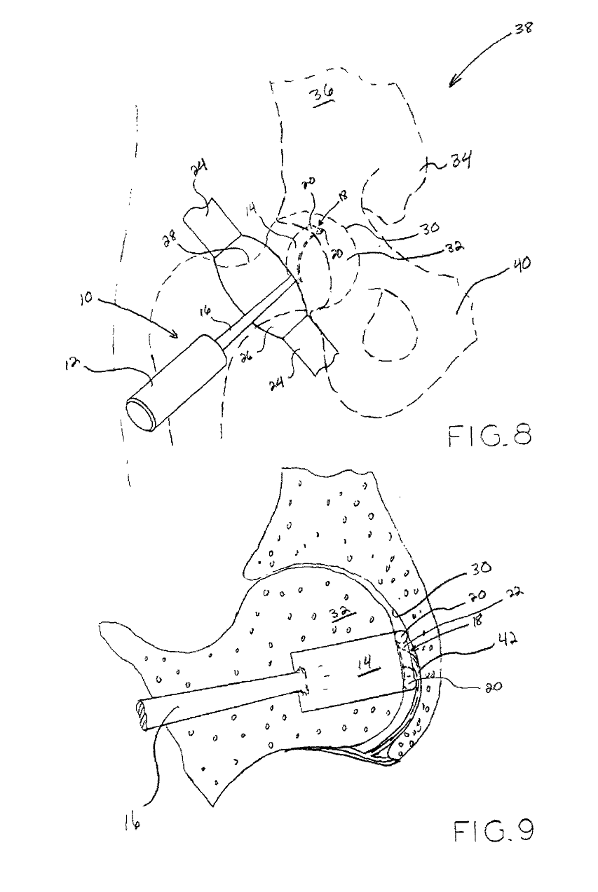

As illustrated in FIGS. 1 and 3, head 14 of ligamentum teres femoris cutter 10 has a radius of curvature whereby head 14 can be placed intermediate the ball and the socket of a ball-and-socket joint as illustrated, e.g., in FIGS. 8 and 9. FIG. 1 illustrates a first exemplary embodiment of ligamentum teres femoris cutter 10 in accordance with the present invention in which extension 16 extends substantially radially from head 14, i.e., along a radius of head 14. FIG. 6 illustrates an alternative embodiment of a ligamentum teres femoris cutter in accordance with the present invention. Ligamentum teres femoris cutter 10′ illustrated in FIG. 6 is constructed whereby extension 16 extends from head 14 in a direction about thirty degrees away from the radius of curvature intersecting the junction of extension 16 and head 14 toward the interior, or concave surface of head 14. In other words, a ray originating from the center point of a radius of curvature of head 14 which intersects the junction of head 14 and the longitudinal axis of extension 16 will form an angle of about thirty degrees with the longitudinal axis of extension 16. As illustrated in FIG. 6, ray 44 forms the angle φ with longitudinal axis 46 of extension 16, with φ measuring about thirty degrees. It is contemplated that various angles will be formed between extension 16 and head 14.

FIGS. 7-9 illustrate use of ligamentum teres femoris cutter 10 to sever ligamentum teres femoris 42 (FIG. 9). As illustrated in FIG. 7, ligamentum teres femoris cutter 10 is inserted through anterior incision 26 held open by retractors 24. As illustrated in FIG. 7, incision 26 can be made so as to generally expose femoral neck 28. FIGS. 7 and 8 illustrate the general structure of hip 38 including pubis 40, anterior superior iliac spine 34, and ilium 36. FIGS. 7 and 8 further illustrate acetabulum 30, with femoral head 32 positioned therein. As illustrated in FIG. 9, ligamentum teres femoris 42 joins femoral head 32 to acetabulum 30.

Upon insertion of distal end 18 of ligamentum teres femoris cutter 10 through incision 26 as illustrated in FIG. 7, distal end 18 of ligamentum teres femoris cutter 10 is positioned intermediate femoral head 32 and acetabulum 30, as illustrated in FIG. 8. Ligamentum teres femoris cutter 10 is thereafter moved into position as illustrated in FIG. 9, with joint spacers 20 positioned on opposite sides of ligamentum teres femoris 42 at its connection to femoral head 32. With head 14 of ligamentum teres femoris cutter 10 positioned as illustrated in FIG. 9, joint spacers 20 function to extend the joint spacing between femoral head 32 and acetabulum 30 and thereby tension ligamentum teres femoris 42 to facilitate severing thereof. Importantly, joint spacers 20 provide a guide (via tactile feedback) to ensure that ligamentum teres femoris 42 is positioned intermediate joint spacers 20 to allow blade 22 to contact ligamentum teres femoris 42. Ligamentum teres femoris cutter 10 is positioned whereby blade 22 contacts ligamentum teres femoris 42 to sever ligamentum teres femoris 42 from femoral head 32.

The ligamentum teres femoris cutter of the present invention can be formed as either a disposable, or a reusable surgical instrument.

While this invention has been described as having a preferred design, the present invention can be further modified within the spirit and scope of this disclosure. This application is therefore intended to cover any variations, uses, or adaptations of the invention using its general principles. Further, this application is intended to cover such departures from the present disclosure as come within know or customary practice in the art to which this invention pertains and which fall within the limits of the appended claims.

What is claimed is:

1. A ligament cutter, comprising:
a handle;
a head having a distal end with a blade, said head connected to said handle; and
spacer means connected to said head for extending a spacing between a pair of bones forming a joint and thereby tensioning a ligament joining said pair of bones.

2. The ligament cutter of claim 1, wherein said spacer means comprises a pair of joint spacers, said blade spanning said joint spacers, said joint spacers having a thickness whereby said joint spacers are operable to extend a joint space between a femoral head and an acetabulum to thereby tension a ligamentum teres femoris.

3. The ligament cutter of claim 2, wherein said thickness is about 31 millimeters.

4. The ligament cutter of claim 1, wherein said head has a radius of curvature whereby said head can be positioned intermediate a ball and a socket of a ball-and-socket joint.

5. The ligament cutter of claim 4, wherein said handle is connected to said head via an extension, said extension extending substantially radially from said head.

6. The ligament cutter of claim 4, wherein said handle is connected to said head via an extension, said extension extending from said head in a direction about 30° away from said radius of curvature toward a concave surface of said head.

7. The ligament cutter of claim 1, wherein said blade is recessed from said spacer means.

8. A ligament cutter, comprising:
a handle;
a head having a distal end with a blade, said head connected to said handle; and
a pair of joint spacers, said blade spanning said joint spacers, said joint spacers having a thickness whereby said joint spacers are operable to extend a joint space between a femoral head and an acetabulum to thereby tension a ligamentum teres femoris.

9. The ligament cutter of claim 8, wherein said thickness is about 31 millimeters.

10. The ligament cutter of claim 8, wherein said head has a radius of curvature whereby said head can be positioned intermediate a ball and a socket of a ball-and-socket joint.

11. The ligament cutter of claim 10, wherein said handle is connected to said head via an extension, said extension extending substantially radially from said head.

12. The ligament cutter of claim 10, wherein said handle is connected to said head via an extension, said extension extending from said head in a direction about 30° away from said radius of curvature toward a concave surface of said head.

13. The ligament cutter of claim 8, wherein said blade is recessed from said joint spacers.

14. A ligament cutter, comprising:
a cylindrical handle having grip means for facilitating a firm grip of the ligament cutter;
an extension connected to said handle, said extension tapering away from said handle; and
a head connected to said extension, said head having a distal end with a pair of joint spacers connected thereto, said blade spanning said joint spacers, said joint spacers having a thickness which is greater than a thickness of said blade, wherein said head has a radius of curvature whereby said head can be positioned intermediate a ball and a socket of a ball-and-socket joint;
wherein said handle is connected to said head via an extension, said extension extending substantially radially from said head;
wherein said blade is recessed from said joint spacers.

15. A ligament cutter, comprising:
a handle;
a head having a distal end with a blade, said head connected to said handle; and
a pair of joint spacers, said blade spanning said joint spacers, wherein said blade is recessed from said joint spacers.

16. The ligament cutter of claim 15, wherein said head has a radius of curvature whereby said head can be positioned intermediate a ball and a socket of a ball-and-socket joint.

17. The ligament cutter of claim 10, wherein said handle is connected to said head via an extension, said extension extending substantially radially from said head.

18. The ligament cutter of claim 15, wherein said handle is connected to said head via an extension, said extension extending from said head in a direction about 30° away from said radius of curvature toward a concave surface of said head.
